(19) United States
(12) Patent Application Publication
(10) Pub. No.: US 2026/0248179 A1
(43) Pub. Date: Aug. 27, 2026

(54) AEROSOL GENERATING DEVICE

(71) Applicant: KT&G CORPORATION, Daejeon (KR)

(72) Inventors: Won Kyeong LEE, Gyeonggi-do (KR); Min Kyu KIM, Seoul (KR)

(73) Assignee: KT&G CORPORATION, Daejeon (KR)

(21) Appl. No.: 18/993,061

(22) PCT Filed: Oct. 12, 2023
(86) PCT No.: PCT/KR2023/015717

§ 371 Date: Jan. 10, 2025

(30) Foreign Application Priority Data
Dec. 2, 2022 (KR) 10-2022-0167064
Feb. 14, 2023 (KR) 10-2023-0019597

(51) Int. Cl. A24F 40/30; A24F 40/10; A24F 40/42; A24F 40/44; A24F 40/50

(52) U.S. Cl. CPC A24F 40/30; A24F 40/10; A24F 40/42; A24F 40/44; A24F 40/50

(57) ABSTRACT

An aerosol generating device includes a storage including a plurality of capsules storing an aerosol generating material, an atomizer configured to generate an aerosol from the aerosol generating material, and a rupture unit configured to rupture the capsules and supply the aerosol generating material stored in the capsules to the atomizer, wherein the storage is movable such that the capsules are aligned at a location corresponding to the rupture unit.

### TECHNICAL FIELD

One or more embodiments relate to an aerosol generating device, and more particularly, to an aerosol generating device in which an aerosol generating material is supplied through the breakage of a capsule.

### BACKGROUND ART

Recently, research on aerosol generating devices is being actively conducted. An aerosol generating device is a device for generating an aerosol from a liquid or solid aerosol generating material or generating vapor from a liquid aerosol generating material and then allowing the generated vapor to pass through a solid-state aromatic medium to provide an aerosol with a flavor.

Examples of fields to which aerosol generating devices are applied include electronic cigarettes for generating an aerosol by heating a cigarette or a liquid composition, medication inhalation devices for aerosolizing pharmaceutical compounds for the treatment of diseases, such as asthma or lung diseases, and the like.

In the field of aerosol generating devices using liquid-state aerosol generating materials, research on the prevention of liquid leakage and quantitative supply of liquids has been actively conducted.

### DISCLOSURE OF INVENTION

### Technical Problem

A cartridge of an aerosol generating device generally includes a storage storing therein a liquid material (hereinafter, the liquid material may be used interchangeably with “aerosol generating material” and is shortly referred to as “liquid”), and an atomizer for atomizing a liquid. The atomizer may include a wick (e.g., cotton, silica, ceramic, etc.) for absorbing a liquid and a heating element (e.g., a coil according to a resistance heating principle) for atomizing the liquid. The liquid stored in the storage may be delivered to the wick and heated by the heating element coupled to the wick, thereby being converted into an aerosol.

In a structure in which the liquid is transferred from the storage to the wick, the liquid may leak from a portion in which the storage is coupled to the wick. Also, while the liquid is transferred from the storage to a portion of the wick that is adjacent to the heating element, the liquid may leak because of backflow.

According to one or more embodiments, there is provided an aerosol generating device including one or more capsules for supplying an aerosol generating material to an atomizer only upon the rupture of capsules containing the aerosol generating material.

In addition, according to one or more embodiments, an aerosol generating device is provided with a structure in which another unruptured capsule may be ruptured while retaining the previously ruptured capsule.

The technical problems of the present disclosure are not limited to the above-described description, and other technical problems may be clearly understood by one of ordinary skill in the art from the embodiments to be described hereinafter.

### Solution to Problem

An aerosol generating device according to an embodiment includes a storage including a plurality of capsules storing an aerosol generating material, an atomizer configured to generate an aerosol from the aerosol generating material, and a rupture unit configured to rupture the capsules and supply the aerosol generating material stored in the capsules to the atomizer, wherein the storage is movable such that the capsules are aligned at a location corresponding to the rupture unit.

### Advantageous Effects of Invention

According to an aerosol generating device according to one or more embodiments, leakage of an aerosol generating material may be prevented.

Also, according to aerosol generating device according to one or more embodiments, because the amount of aerosol generating material released from capsules is uniform whenever the capsules are ruptured, the aerosol generating material may be quantitatively provided to an atomizer, and thus, the uniform amount of aerosol may be provided to a user with each use.

Effects of the present disclosure are not limited to the above effects, and effects that are not mentioned could be clearly understood by one of ordinary skill in the art from the present specification and the attached drawings.

### BRIEF DESCRIPTION OF DRAWINGS

FIG. 1 is a longitudinal cross-sectional view of an aerosol generating device according to an embodiment.

FIGS. 2A and 2B respectively are perspective views illustrating examples of a storage applicable to the aerosol generating device according to the embodiment of FIG. 1.

FIGS. 3A to 3D respectively are cross-sectional views illustrating examples of a rupture unit applicable to the aerosol generating device according to the embodiment of FIG. 1.

FIGS. 4A to 4C respectively are cross-sectional views illustrating examples in which an atomizer and a rupture unit are coupled to each other in a structure applicable to the aerosol generating device according to the embodiment of FIG. 1.

FIG. 5A is a cross-sectional view of an atomizer and a rupture unit, illustrating an example of the rupture unit in a first operation state.

FIG. 5B is a cross-sectional view of an atomizer and a rupture unit, illustrating the rupture unit of FIG. 5A in a second operation state.

FIGS. 6A to 6D are cross-sectional views illustrating, in sequence, a capsule processing process of an aerosol generating device, according to an embodiment.

FIG. 7 is a block diagram of an aerosol generating device according to an embodiment.

FIG. 8 is a flowchart illustrating a capsule processing process of an aerosol generating device, according to an embodiment.

FIG. 9 is a block diagram of an aerosol generating device according to another embodiment.

### MODE FOR THE INVENTION

Regarding the terms in the various embodiments, the general terms which are currently and widely used are selected in consideration of functions of structural elements in the various embodiments of the present disclosure. However, meanings of the terms can be changed according to intention, a judicial precedence, the appearance of a new technology, and the like. In addition, in certain cases, terms which can be arbitrarily selected by the applicant in particular cases. In such a case, the meaning of the terms will be described in detail at the corresponding portion in the description of the present disclosure. Therefore, the terms used in the various embodiments of the present disclosure should be defined based on the meanings of the terms and the descriptions provided herein.

In addition, unless explicitly described to the contrary, the word “comprise” and variations such as “comprises” or “comprising” will be understood to imply the inclusion of stated elements but not the exclusion of any other elements. In addition, the terms “-er”, “-or”, and “module” described in the specification mean units for processing at least one function and operation and can be implemented by hardware components or software components and combinations thereof.

As used herein, when an expression such as “at least any one” precedes arranged elements, it modifies all elements rather than each arranged element. For example, the expression “at least any one of a, b, and c” should be construed to include a, b, c, or a and b, a and c, b and c, or a, b, and c.

In an embodiment, an aerosol generating device may be a device that generates aerosols by electrically heating a cigarette accommodated in an interior space thereof.

The aerosol generating device may include a heater. In an embodiment, the heater may be an electro-resistive heater. For example, the heater may include an electrically conductive track, and the heater may be heated when currents flow through the electrically conductive track.

The heater may include a tube-shaped heating element, a plate-shaped heating element, a needle-shaped heating element, or a rod-shaped heating element, and may heat the inside or outside of a cigarette according to the shape of a heating element.

A cigarette may include a tobacco rod and a filter rod. The tobacco rod may be formed of sheets, strands, and tiny bits cut from a tobacco sheet. Also, the tobacco rod may be surrounded by a heat conductive material. For example, the heat conductive material may be, but is not limited to, a metal foil such as aluminum foil.

The filter rod may include a cellulose acetate filter. The filter rod may include at least one segment. For example, the filter rod may include a first segment configured to cool aerosols, and a second segment configured to filter a certain component in aerosols.

In another embodiment, the aerosol generating device may be a device that generates aerosols by using a cartridge containing an aerosol generating material.

The aerosol generating device may include a cartridge that contains an aerosol generating material, and a main body that supports the cartridge. The cartridge may be detachably coupled to the main body, but is not limited thereto. The cartridge may be integrally formed or assembled with the main body, and may also be fixed to the main body so as not to be detached from the main body by a user. The cartridge may be mounted on the main body while accommodating an aerosol generating material therein. However, the present disclosure is not limited thereto. An aerosol generating material may also be injected into the cartridge while the cartridge is coupled to the main body.

The cartridge may contain an aerosol generating material in any one of various states, such as a liquid state, a solid state, a gaseous state, a gel state, or the like. The aerosol generating material may include a liquid composition. For example, the liquid composition may be a liquid including a tobacco-containing material having a volatile tobacco flavor component, or a liquid including a non-tobacco material.

The cartridge may be operated by an electrical signal or a wireless signal transmitted from the main body to perform a function of generating aerosols by converting the phase of an aerosol generating material inside the cartridge into a gaseous phase. The aerosols may refer to a gas in which vaporized particles generated from an aerosol generating material are mixed with air.

In another embodiment, the aerosol generating device may generate aerosols by heating a liquid composition, and generated aerosols may be delivered to a user through a cigarette. That is, the aerosols generated from the liquid composition may move along an airflow passage of the aerosol generating device, and the airflow passage may be configured to allow aerosols to be delivered to a user by passing through a cigarette.

In another embodiment, the aerosol generating device may be a device that generates aerosols from an aerosol generating material by using an ultrasonic vibration method. At this time, the ultrasonic vibration method may mean a method of generating aerosols by converting an aerosol generating material into aerosols with ultrasonic vibration generated by a vibrator.

The aerosol generating device may include a vibrator, and generate a short-period vibration through the vibrator to convert an aerosol generating material into aerosols. The vibration generated by the vibrator may be ultrasonic vibration, and the frequency band of the ultrasonic vibration may be in a frequency band of about 100 kHz to about 3.5 MHz, but is not limited thereto.

The aerosol generating device may further include a wick that absorbs an aerosol generating material. For example, the wick may be arranged to surround at least one area of the vibrator, or may be arranged to contact at least one area of the vibrator.

As a voltage (for example, an alternating voltage) is applied to the vibrator, heat and/ or ultrasonic vibrations may be generated from the vibrator, and the heat and/or ultrasonic vibrations generated from the vibrator may be transmitted to the aerosol generating material absorbed in the wick. The aerosol generating material absorbed in the wick may be converted into a gaseous phase by heat and/or ultrasonic vibrations transmitted from the vibrator, and as a result, aerosols may be generated.

For example, the viscosity of the aerosol generating material absorbed in the wick may be lowered by the heat generated by the vibrator, and as the aerosol generating material having a lowered viscosity is granulated by the ultrasonic vibrations generated from the vibrator, aerosols may be generated, but is not limited thereto.

In another embodiment, the aerosol generating device is a device that generates aerosols by heating an aerosol generating article accommodated in the aerosol generating device using an induction heating method.

The aerosol generating device may include a susceptor and a coil. In an embodiment, the coil may apply a magnetic field to the susceptor. As power is supplied to the coil from the aerosol generating device, a magnetic field may be formed inside the coil. In an embodiment, the susceptor may be a magnetic body that generates heat by an external magnetic field. As the susceptor is positioned inside the coil and a magnetic field is applied to the susceptor, the susceptor generates heat to heat an aerosol generating article. In addition, optionally, the susceptor may be positioned within the aerosol generating article.

In another embodiment, the aerosol generating device may further include a cradle.

The aerosol generating device may configure a system together with a separate cradle. For example, the cradle may charge a battery of the aerosol generating device. Alternatively, the heater may be heated when the cradle and the aerosol generating device are coupled to each other.

Hereinafter, the present disclosure will now be described more fully with reference to the accompanying drawings, in which exemplary embodiments of the present disclosure are shown such that one of ordinary skill in the art may easily work the present disclosure. The present disclosure may be implemented in a form that can be implemented in the aerosol generating devices of the various embodiments described above or may be implemented in various different forms, and is not limited to the embodiments described herein.

Hereinafter, embodiments of the present disclosure will be described in detail with reference to the drawings.

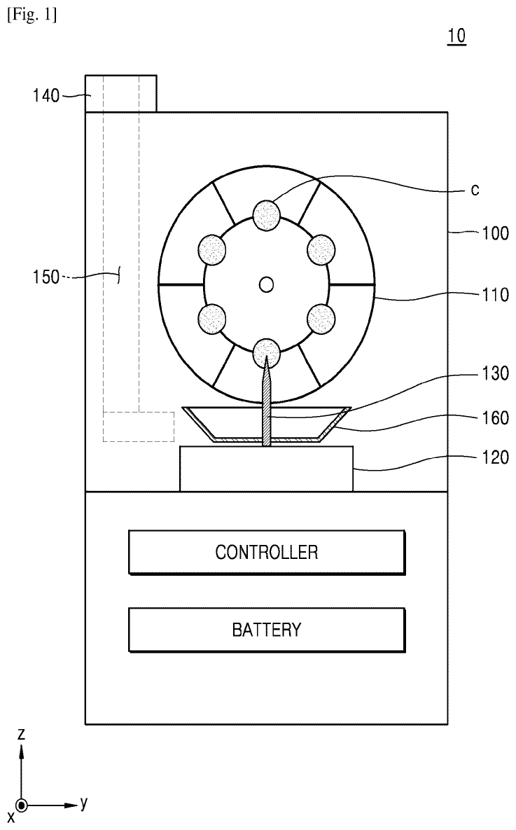

FIG. 1 is a longitudinal cross-sectional view of an aerosol generating device according to an embodiment.

Referring to FIG. 1, an aerosol generating device 10 according to an embodiment includes a storage 110 including a plurality of capsules, an atomizer 120 configured to generate an aerosol, and a rupture unit 130 configured to rupture a capsule to supply an aerosol generating material to the atomizer.

The aerosol generating device 10 may include a housing 100. The housing 100 may form an overall exterior of the aerosol generating device 10. Components of the aerosol generating device 10 may be arranged in an inner space of the housing 100. The housing 100 may accommodate and protect the components.

At least a portion of the housing 100 may be separated from the other portions thereof so that the components in the inner space of the housing 100 may be replaced. For example, an upper portion of the housing 100 may be disengaged from a lower portion thereof and separated therefrom in a +z direction. As another example, at least a surface of the housing 100 may be open. When at least a portion of the housing 100 is separated, the components of the aerosol generating device 10 may be externally exposed.

The storage 110 may be located inside the housing 100 and store an aerosol generating material in a liquid or gel state. For example, in the field of electronic cigarettes, an aerosol generating material may be a liquid including a tobacco-containing material having a volatile tobacco flavor component, or a liquid including a non-tobacco material.

In the aerosol generating device, the aerosol generating material may leak (leakage during storage) in a portion in which the storage is connected to the atomizer. In addition, while the aerosol generating material is transferred from the storage to the atomizer, leakage of the aerosol generating material (usage leakage) may occur because of backflow. With regard to the design of the storage and the atomizer, it is crucial to reduce the occurrence of leakage.

To overcome the above problems, the storage 110 of the aerosol generating device 10 according to an embodiment may include at least one capsule c including the aerosol generating material. The capsule c may store the aerosol generating material, and only when the capsule c is ruptured, the aerosol generating material may be supplied to the atomizer 120.

In this case, the term ‘rupture’ may include various methods of releasing the aerosol generating material stored in the capsule c by damaging an outer surface of the capsule c through crushing or breaking under pressure, cutting with a sharp blade, puncturing with a pointed needle, or the like.

Before the rupture of the capsule c, the aerosol generating material stored in the capsule c may not be discharged to the outside of the capsule c. Hereinafter, a state before the capsule c is ruptured may be referred to as the ‘storage state of a capsule.’

When the capsule c is ruptured by the rupture unit 130, the aerosol generating material may be released from the capsule c and move to the atomizer 120. Hereinafter, a state in which the capsule c is ruptured may be referred to as the ‘ruptured state of a capture.’

According to an embodiment, because the aerosol generating material is supplied to the atomizer 120 only when the capsule c storing the aerosol generating material is ruptured, the leakage, which may occur while the aerosol generating material is stored in the capsule c (prevention of storage leakage), may be prevented.

Also, according to an embodiment, when the capsule c is ruptured, the capsule c may partially lose the function of storing the aerosol generating material. Therefore, while the aerosol generating material moves from the ruptured capsule c to the atomizer 120, the aerosol generating material may be prevented from backflowing (preventing usage leakage).

In addition, according to an embodiment, the capsule c mounted in the storage 110 may not be directly connected to the atomizer 120. Accordingly, a path through which the aerosol generating material backflows from the atomizer 120 to the ruptured capsule c is blocked, and thus, the backflow may be prevented (preventing usage leakage).

The capsule c may include a material that is the same as a liquid capsule that is commonly used in the field of aerosol generating devices. However, the materials of the capsule are not limited thereto. The capsule c may prevent the aerosol generating material from leaking when storing the same, and may include various materials that may be ruptured by the rupture unit 130.

The capsule c may be in various forms. Referring to FIG. 1, the capsule may have a spherical shape. However, the shape of the capsule is not limited thereto and may include various shapes for storing the aerosol generating material.

A plurality of capsules c may be mounted in the storage 110. In an aerosol generating device, a storage generally stores the entire amount of aerosol generating material. On the contrary, in the aerosol generating device 10 according to an embodiment, the aerosol generating material may be distributed and stored in the capsules c, and the capsules c may be mounted in the storage 110.

In the field of electronic cigarettes, in comparison with a pack of cigarettes including 20 existing combustible cigarettes, 20 capsules c may be mounted in the storage 110 as a set. However, the number of capsules c is not limited thereto. FIG. 1 illustrates six capsules c.

In the capsules c, the amount of aerosol generating material may be stored such that the amount of aerosols suitable for the user to inhale during a single use of the aerosol generating device 10 may be generated.

In the field of electronic cigarettes, a capsule c may contain an aerosol generating material that is equal to that included in one existing combustible cigarette. When 20 capsules c form a set, the total amount of aerosol generating material in the 20 capsules c may be 1 ml, and one capsule c may include an aerosol generating material ranging from 0.04 ml to 0.06 ml, specifically, 0.05 ml. However, the amount of aerosol generating material stored in the capsule is not limited to the above example.

According to an embodiment, because the amount of aerosol generating material released from the capsules c is uniform whenever the capsules c are ruptured, the aerosol generating material may be quantitatively provided to the atomizer 120. Therefore, whenever the user uses the aerosol generating device 10, the uniform amount of aerosol may be delivered to the user with each use.

Also, according to an embodiment, because the storage 110 is equipped with multiple capsules c containing the aerosol generating material that is suitable for a single use, the user may avoid the inconvenience of replacing the capsules c every time the aerosol generating device 10 is used.

When there are unruptured capsules c among one or more capsules c mounted in the storage 110, the user may not remove the capsules c and simply put the same in a region of the aerosol generating device 10, and may rupture the unruptured capsules c and use the aerosol generating device 10.

When all the capsules c in the storage 110 are ruptured, the user needs to mount a new capsule c in the storage 110 to use the aerosol generating device 10. This process corresponds to the replacement of a storage (a cartridge) in a general aerosol generating device.

A manner in which the capsules c are arranged in the storage 110 may vary depending on the structure of the storage 110. For example, the capsules c may be arranged in the storage side by side in a direction. As another example, the capsules c may be arranged in the storage 110 in sequence to form a circle.

When the capsules c are arranged side by side instead of being randomly arranged, the capsules c may be ruptured in an order of arrangements. That is, the capsules c may be effectively ruptured.

In this case, to improve the efficiency of the rupture, the capsules c may be ruptured at the same location each time. Accordingly, there is a need to move the capsule c, which is scheduled to be ruptured, to a location where rupture occurs, before the capsule c is ruptured by the rupture unit 130.

The capsule c maybe aligned at the location where the rupture occurs because of the storage 110. In other words, the storage 110, in which at least one capsule c is installed, may be moved so that the capsules c are aligned at a location corresponding to the rupture unit 130. In this case, the expression ‘location corresponding to the rupture unit 130’ may refer to a location at which the capsule is ruptured.

As an example of a method of moving the storage 110, the storage 110 may rotate. Referring to FIG. 1, the storage 110 may rotate around a rotation axis extending in the x-axis direction. When the storage 110 rotates, the capsules c mounted in the storage 110 may rotate and move with respect to the rotation axis.

Referring to FIG. 1, the location, at which the capsule c is ruptured, may be the closest to the rupture unit 130 in a circular trajectory of the capsule c resulting from the rotation of the storage 110, and may be the location aligned with the rupture unit 130 in a direction of rupture (e.g., the z-axis direction) by the rupture unit 130 to facilitate the rupture of the capsule c.

When the storage 110 rotates, the capsule c, which has most recently been ruptured by the rupture unit 130, may move further away from the rupture unit 130 along the trajectory of the capsule c, and other capsules c may come close to the location where the rupture occurs, along the trajectory of the capsule c. When the capsule c is aligned at the location corresponding to the rupture unit 130, the capsule c may be ruptured by the rupture unit 130.

The atomizer 120 may generate an aerosol from the aerosol generating material. In detail, as the capsule c mounted in the storage 110 is ruptured, the aerosol generating material may be provided to the atomizer 120 arranged on a lower portion of the storage 110 (e.g., in the −z direction). The aerosol generating material provided to the atomizer 120 may be atomized into an aerosol by the atomizer 120.

The aerosol refers to a suspension of liquid and/or solid fine particles dispersed in the air. Therefore, the aerosol generated from the atomizer 120 may be a state in which vaporized particles generated from the aerosol generating material are mixed with air.

The atomizer 120 may convert the phase of the aerosol generating material into a gaseous phase through vaporization and/or sublimation. That is, the atomizer 120 may generate an aerosol by finely granulating an aerosol generating material in any one of a liquid state, a solid state, and a gel state, or a combination thereof.

Although not specifically illustrated in FIG. 1, the atomizer 120 may include a liquid delivery element coupled to a component of the aerosol generating device 10 and configured to absorb the aerosol generating material, and an atomizing element arranged in the liquid delivery element and configured to atomize the aerosol.

The liquid delivery element may receive the aerosol generating material discharged from the ruptured capsule c and absorb the aerosol generating material. For example, the liquid delivery element may be a wick such as cotton fiber, ceramic fiber, glass fiber, or porous ceramic, but is not limited thereto.

The liquid delivery element may have an elongated shape. For example, the liquid delivery element may have a pillar shape extending in a direction. In detail, the liquid delivery element may have a poly-prism shape, such as a cylindrical shape, rectangular pillar shape, a triangular pillar shape, but the shape is not limited thereto. The liquid delivery element may have a shape that is substantially a rod or a needle.

According to an embodiment, the aerosol generating material absorbed into a portion (e.g., an upper portion) of the liquid delivery element may move to another portion (e.g., a lower portion) of the liquid delivery element according to capillarity. Accordingly, the liquid delivery element may deliver the aerosol generating material to the atomizing element.

The atomizing element may generate an aerosol from the aerosol generating material absorbed into the liquid delivery element. For example, the atomizing element may be a heating element configured to heat the aerosol generating material by generating heat. When the aerosol generating material contacting the heating element is heated by the heating element, the aerosol may be generated from the aerosol generating material.

The heating element may be a metal heating wire, a metal hot plate, a ceramic heater, or the like, but is not limited thereto. The heating element may include a resistor having a temperature coefficient of resistance (TCR). For example, the heating element may include SUS316L.

The heating element may include a conductive filament, such as nichrome wire, and may be heated by a current supply. In addition, the heating element may include a susceptor material heated by an induced magnetic field and may be heated by an induced magnetic field generated from an induction coil arranged separately from the heating element.

As another example, the atomizing element may be an ultrasonic vibrator configured to generate an aerosol from an aerosol generating material according to ultrasonic vibration method. The ultrasonic vibration method may refer to a method of generating aerosols by atomizing an aerosol generating material with ultrasonic vibration generated by a vibrator.

A method in which the atomizing element generates an aerosol is not limited thereto, and may include various methods of generating aerosols from an aerosol generating material.

The atomizing element may be arranged in the liquid delivery element through not only structural connections, such as being wound around the liquid delivery element, but also through a permanent or reversible attachment to the liquid delivery element, for example, spreading, spraying, deposition, plating, immersion, painting, printing, three-dimensional printing, the utilization of instruments. Also, the heating element may be arranged in the liquid delivery element in a manner that, for example, the atomizing element is sintered together during the manufacture of the liquid delivery element.

However, the arrangement of the atomizing element is not limited thereto, and may include various ways in which the atomizing element is arranged in the liquid delivery element while maintaining functions thereof.

Although not specifically illustrated in FIG. 1, at least a portion of the atomizer 120 may be coupled to a component of the aerosol generating device 10 and supported.

The rupture unit 130 may rupture the capsule c and supply the aerosol generating material stored in the capsule c to the atomizer 120.

The rupture unit 130 may be arranged between the storage 110 and the atomizer 120. At least a portion of the rupture unit 130 may be coupled to the atomizer 120 and supported. The rupture unit 130 may be arranged in a direction extending towards the storage 110 from the atomizer 120.

When a capsule c, which is scheduled to be ruptured, is moved by the storage 110 and aligned at the location corresponding to the rupture unit 130, the rupture unit 130 may rupture the capsule c, which is scheduled to be ruptured, at the location where the rupture occurs.

The rupture unit 130 may have an elongated shape. An end portion of the rupture unit 130 may be pointed towards the capsule c so that the capsule c may be ruptured. The rupture unit 130 may pierce the capsule c using the pointed end portion of the rupture unit 130 so that the capsule c may be ruptured. However, the shape and rupture method of the rupture unit 130 are not limited thereto.

The aerosol generating material stored in the capsule c may be released to the outside of the capsule c through the portion of the capsule c that is ruptured by the rupture unit 130. The aerosol generating material released from the capsule c may be moved in the gravity direction (e.g., the −z direction) due to gravity and supplied to the atomizer 120.

For example, the aerosol generating material may fall free towards the atomizer 120 and may be supplied to the atomizer 120. As another example, the aerosol generating material may be moved along the rupture unit 130 and then supplied to the atomizer 120. However, the method in which the aerosol generating material released from the capsule c is supplied to the atomizer 120 is not limited to the above embodiments.

To rupture the capsule c, the capsule c needs to come close to the rupture unit 130. For example, at least a portion of the storage 110 may be moved towards the rupture unit 130, and thus, the capsule c may approach the rupture unit 130. As another example, at least a portion of the rupture unit 130 may be moved towards the storage 110, and thus, the rupture unit 130 may approach the capsule c.

The method in which the capsule c approaches the rupture unit 130 is not limited to the above examples. Hereinafter, the rupture of the capsule c is described based on the method in which the rupture unit 130 comes close to the capsule c.

At least a portion of the rupture unit 130 may approach the capsule c aligned at the location where the rupture occurs. That is, at least a portion of the rupture unit 130 may be moved towards the location at which the rupture unit 130 contacts the capsule c.

For example, the rupture unit 130 may be coupled to the atomizer 120 and moved together with the atomizer 120. As another example, although the rupture unit 130 is coupled to the atomizer 120, the rupture unit 130 may only move without the atomizer 120.

According to an embodiment, at least a portion of the rupture unit 130 may move between the location in contact with the capsule c and the location spaced apart from the capsule c.

When at least a portion of the rupture unit 130 comes close to the capsule c and contacts an outer shell of the capsule c, the capsule c may be ruptured through a pointed portion of the at least a portion of the rupture unit 130.

At least a portion of the rupture unit 130 may further move after contacting the outer shell of the capsule c and thus may be inserted into the capsule c. That is, the expression ‘location in contact with the capsule’ may refer to not only a location where at least a portion of the rupture unit 130 contacts the outer shell of the capsule c but a location where at least a portion of the rupture unit 130 is inserted into the capsule c.

As a result, when the rupture unit 130 contacts the outer shell of the capsule c, the capsule c may not be ruptured, or even if a small area is ruptured which is insufficient for the release of the aerosol generating material, the capsule c is ruptured during a process of inserting the rupture unit 130 into the capsule c; thus, a complete rupture of the capsule c may be guaranteed.

When the rupture unit 130 finishes rupturing the capsule c, at least a portion of the rupture unit 130 may be moved in a direction that is opposite to a direction facing the ruptured capsule c and thus may be further away from the ruptured capsule c. That is, at least a portion of the rupture unit 130 may be moved in a direction away from the capsule c.

A mouthpiece 140 may be located on an upper portion of the housing 100 and contact the user's mouth. The mouthpiece 140 may protrude from the housing 100 in the +z direction. The mouthpiece 140 may be shaped to be in easy contact with the user's mouth. The user may put his/her mouth on the mouthpiece 140 formed on the housing 100 and then inhale the aerosol.

An airflow path 150 may serve as a passage through which the atomized aerosol is delivered to the user. In general, an airflow path may refer to a path of the air and aerosol to the mouthpiece 140 from an air inlet (not illustrated) arranged on a portion of the housing 100, but in the present specification, the airflow path may refer to a path through which the aerosol atomized by the atomizer 120 moves to the mouthpiece 140.

External air may flow into the aerosol generating device 10 through the air inlet. The external air may move along an airflow passage (not illustrated) formed inside the aerosol generating device 10 and reach the atomizer 120. The air reaching the atomizer 120 may be mixed with vaporized particles generated from the aerosol generating material. The mixed aerosol may move along the airflow path 150 and may be provided to the user through the mouthpiece 140.

According to an embodiment, the airflow path 150 may be arranged to extend from the atomizer 120 to the mouthpiece 140 by bypassing the storage 110. However, the arrangement of the airflow path is not limited thereto.

The aerosol generating device 10 according to an embodiment may further include an accommodation portion 160. The accommodation portion 160 may be arranged on a lower portion of the storage 110 and accommodate the aerosol generating material released from the capsule c that is ruptured by the rupture unit 130.

The accommodation portion 160 may include a funnel shape that is open in the z-axis direction. An upper portion of the funnel facing the ruptured capsule c may be open enough to allow the aerosol generating material released from the capsule c to be introduced to the inside of the accommodation portion 160. A lower portion of the funnel facing the atomizer 120 may be open to the extent that the aerosol generating material introduced to the accommodation portion 160 descends and is completely supplied to the atomizer 120.

The rupture unit 130 may be arranged inside the accommodation portion 160 having the funnel shape that is open in the z-axis direction. In this case, the accommodation portion 160 may allow the storage 110 and/or the rupture unit 130 to move such that the rupture unit 130 may rupture the capsule c.

The material forming the accommodation portion 160 may include various materials allowing the aerosol generating material introduced to the inside of the accommodation portion 160 not to remain in the accommodation portion 160 and to actively move along an inner side surface thereof.

According to an embodiment, the accommodation portion 160 may prevent the aerosol generating material from descending around the atomizer 120 and guide the aerosol generating material released from the ruptured capsule c to completely move to the atomizer 120.

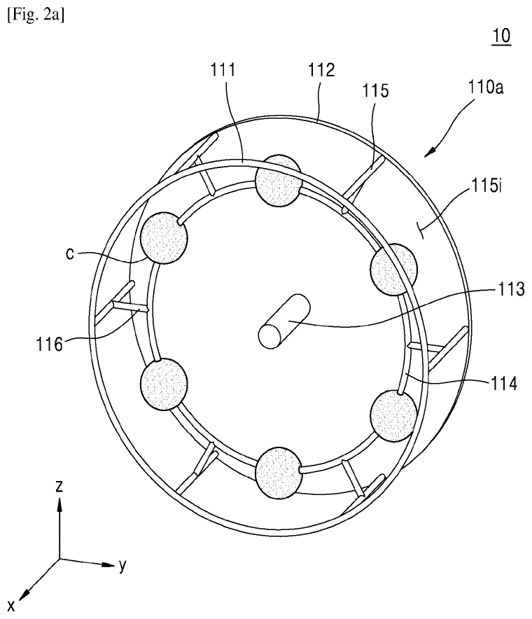

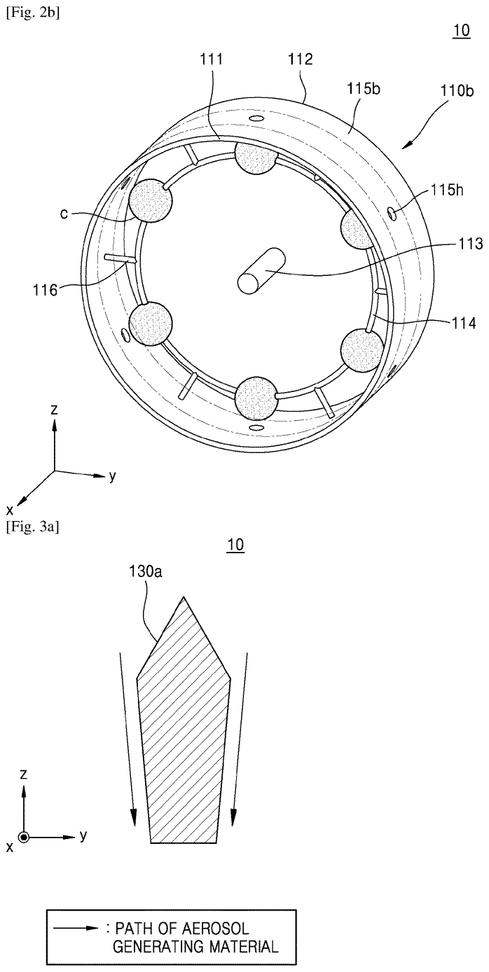

Hereinafter, the specific structure of the storage 110 including the capsule c and the movement of the storage 110 to align a capsule at a location where rupture occurs are described with reference to FIGS. 2A and 2B.

FIGS. 2A and 2B respectively are perspective views illustrating examples of a storage applicable to the aerosol generating device according to the embodiment of FIG. 1.

Referring to FIGS. 2A and 2B, the aerosol generating device 10 according to an embodiment may include storages 110a and 110b. The storage 110a of FIG. 2A and the storage 110b of FIG. 2B may be the same as or similar to the storage 110 of FIG. 1, and repeated descriptions are omitted.

Referring to FIG. 2A, the storage 110a may include a front portion 111, a rear portion 112, a central portion 113, a mounting portion 114, a connector 115, and a support 116.

The front portion 111 and the rear portion 112 may form the exterior of the storage 110a. The front portion 111 may include a circular frame and may be open in the x-axis direction. The user may replace one or more used capsules c through the open portion of the front portion 111. The rear portion 112 may include a circular plate and be connected to the central portion 113 of the storage 110a.

The front portion 111 and the rear portion 112 may be arranged side by side in a direction crossing the x-axis direction (e.g., a direction in which a y-z plane extends). In this case, the front portion 111 and the rear portion 112 may have the same size in the direction crossing the x-axis direction. However, the arrangements and shapes of the front portion 111 and the rear portion 112 are not limited to the embodiments described above.

The central portion 113 may be arranged at the center of the circular storage 110a when the storage 110a is viewed in the x-axis direction. The central portion 113 may be coupled to the rear portion 112 through the center of the rear portion 112.

Although not specifically illustrated in FIG. 1, the central portion 113 may be coupled to a component of the aerosol generating device 10 and supported. To this end, the storage 110a may be supported by a component of the aerosol generating device 10 inside the aerosol generating device 10.

The central portion 113 may include the rotation axis extending in the x-axis direction. The central portion 113 may rotate around the rotation axis by a driver (not illustrated). When the central portion 113 rotates, the rear portion 112 coupled to the central portion 113 may rotate together with the central portion 113. Accordingly, the storage 110a may rotate around the central portion 113 taken as the rotation axis.

At least one capsule c may be mounted in the mounting portion 114. The mounting portion 114 may include a circular frame in which the capsules c form a circle and are arranged in the storage 110a at the same intervals. The mounting portion 114 may support at least one capsule c mounted.

For example, the capsules c may be mounted in the mounting portion 114 one by one. As another example, the capsules c may form a set connected to adjacent capsules c. In this case, the user may mount one set of capsules c in the storage 110a without separating the set of capsules c into respective capsules c.

The mounting portion 114 may be arranged between the front portion 111 and the rear portion 112 in parallel therewith. In this case, the mounting portion 114 may have a size that is less than sizes of the front portion 111 and the rear portion 112 in a direction crossing the x-axis direction.

As an example of a method of mounting the capsule c in the mounting portion 114, the mounting portion 114 may include a hole (not illustrated) corresponding to the size of the capsule c. The capsule c may be coupled to the hole in a fitting manner. However, the method of mounting the capsule c in the mounting portion 114 is not limited to the above example, and various methods may be included in which the capsule c may be ruptured by the rupture unit 130 and supported by the mounting portion 114.

The connector 115 may connect the front portion 111 to the rear portion 112. The support 116 may connect the mounting portion 114 to the connector 115. Because the rear portion 112 is coupled to the central portion 113, all components of the storage 110a may be connected to each other and rotate together. Accordingly, one or more capsules c mounted in the mounting portion 114 may rotate and move together with the storage 110a. In this case, the circular mounting portion 114 may be the trajectory of the capsule c.

The connector 115 and the support 116 may each include a straight frame. The connector 115 may firmly support the front portion 111 and the rear portion 112. The support 116 may firmly support the mounting portion 114 and the connector 115.

The connector 115 may separate the front portion 111 from the rear portion 112 by as much as the connector 115 extends. Accordingly, a space 115i corresponding to the extension of the connector 115 may be formed. The space 115i may be a gap between the front portion 111 and the rear portion 112 and between one or more connectors 115.

The connector 115 may be arranged not to interfere with the path through which the rupture unit (e.g., the rupture unit 130 of FIG. 1) travels to rupture the capsule c. Therefore, the space 115i may become a space where the rupture unit moves to rupture the capsule c and may allow the rupture unit to move. Also, the space 115i may include a space great enough for the aerosol generating material released from the capsule c to move to the atomizer 120.

Referring to FIG. 2B, the storage 110b may include the front portion 111, the rear portion 112, the central portion 113, the mounting portion 114, and the support 116, similarly to the storage 110a of FIG. 2A.

Unlike the storage 110a of FIG. 2A, the storage 110b of FIG. 2B may include a side portion 115b. In this case, the support 116 may connect the mounting portion 114 to the side portion 115b and firmly support the two.

The side portion 115b may form the exterior of the storage 110b. The side portion 115b may have a cylindrical shape that is open in the x-axis direction and connect the front portion 111 to the rear portion 112 between the front portion 111 and the rear portion 112. Accordingly, the storage 110b may have a cylindrical exterior that is open in the +x direction, only in the front portion 111.

The side portion 115b may include an insertion hole 115h guiding the rupture unit that moves towards the capsule aligned at the location corresponding to the rupture unit. The insertion hole 115h may prevent the rupture unit from failing to rupture the capsule c because of the deviation of the rupture unit from the movement path thereof towards the capsule.

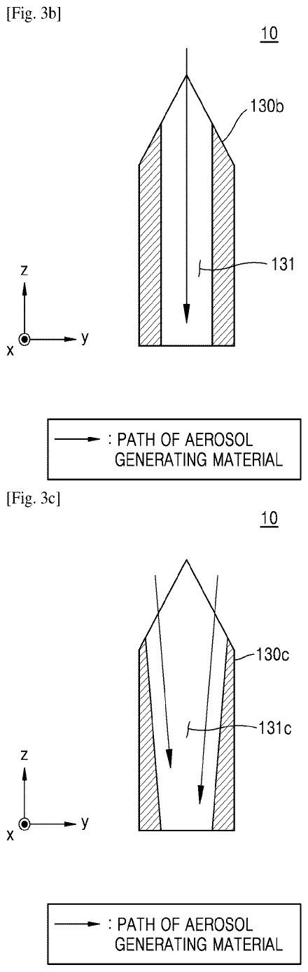

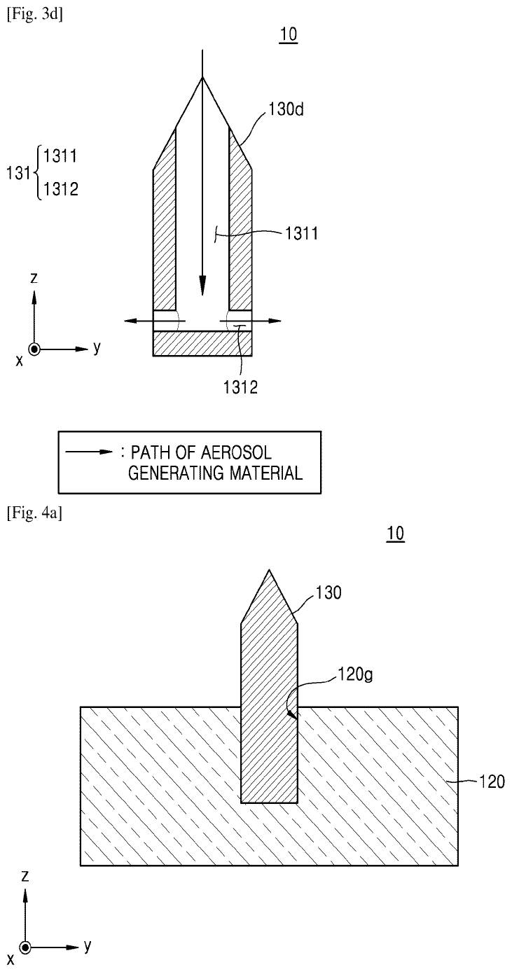

Hereinafter, with reference to FIGS. 3A to 3D, various shapes of the rupture unit capable of guiding the movement of the aerosol generating material are described.

FIGS. 3A to 3D respectively are cross-sectional views illustrating examples of a rupture unit applicable to the aerosol generating device of FIG. 1.

Referring to FIGS. 3A to 3D, the aerosol generating device 10 according to an embodiment may include rupture units 130a, 130b, 130c, and 130d. The rupture units respectively illustrated in FIGS. 3A to 3D may be the same as or similar to the rupture unit 130 of FIG. 1, and repeated descriptions may be omitted, and the common descriptions are provided based on the rupture unit 130 of FIG. 1.

Referring to FIGS. 3A to 3D, the rupture units 130a to 130d may not only release the aerosol generating material by simply rupturing the capsule (e.g., the capsule c of FIG. 1) but may each serve as a medium for delivering the aerosol generating material.

Referring to FIG. 3A, the rupture unit 130a may have a shape in which at least a portion of the rupture unit 130a is inclined in a direction in which the rupture unit 130a extends (hereinafter, a lengthwise direction of the rupture unit). For example, when the rupture unit 130a with an elongated shape is arranged in the z-axis direction, at least a portion of the rupture unit 130a may be inclined with respect to the z-axis direction. In this case, the cross-section of the rupture unit 130a in a longitudinal direction (e.g., the z-axis direction) may have a pointed shape or a tapered shape.

Because of the inclined portion of the rupture unit 130a, the rupture unit 130a may have a shape in which the thickness of the rupture unit 130a decreases towards an end portion thereof. In this case, the term “thickness” may refer to the distance from the central axis of the rupture unit (hereinafter, the expression “central axis of the rupture unit” may be used in this sense) extending in the lengthwise direction (e.g., the z-axis direction) of the rupture unit to a point on the circumference of the rupture unit along a radial direction of the rupture unit.

Like the rupture unit 130 of FIG. 1, the rupture unit 130a may be inclined in a direction towards the storage (e.g., the storage 110 of FIG. 1). For example, an end portion of the rupture unit 130a may have a pointed shape. The rupture unit 130a may pierce the capsule c using the pointed end portion of the rupture unit 130a so that the capsule c may be ruptured.

Also, the rupture unit 130a may be inclined in a direction towards the atomizer (e.g., the atomizer 120 of FIG. 1). For example, the rupture unit 130a may be inclined in a direction towards the other end portion opposite to the end portion facing the storage. The rupture unit 130a may decrease in thickness towards the other end portion thereof.

After the rupture unit 130 of FIG. 1 ruptures the capsule c, the end portion of the rupture unit 130 may remain inserted into the capsule. In this case, the ruptured portion of the capsule may be sealed by a region of the rupture unit 130. Therefore, the release of the aerosol generating material through the ruptured portion may be difficult.

In this case, when the rupture unit 130a of FIG. 3A decreases in thickness towards the other end portion thereof, there may be a space where the aerosol generating material may be released by the rupture unit 130a having the ruptured portion with a decreasing thickness as the rupture unit 130a moves towards the inside of the capsule. Therefore, the aerosol generating material may be smoothly released through a wide clearance.

The external surface of the rupture unit 130a may serve to guide the aerosol generating material to move towards the atomizer. The aerosol generating material effectively released may fall free towards the atomizer and move towards the atomizer along the external surface of the rupture unit 130a.

Referring to FIG. 3B, the rupture unit 130b may include a hollow hole 131 extending in a lengthwise direction of the rupture unit 130b. When the rupture unit 130b remains inserted into the capsule after breaking the same, the aerosol generating material may move towards the atomizer through the hollow hole 131 that is open towards the atomizer.

When the aerosol generating material moves through the hollow hole 131, the possibility that the aerosol generating material reaches the atomizer may increase compared to a case where the aerosol generating material is simply released through the ruptured portion of the capsule and moves. Accordingly, the arrangement of the hollow hole 131 may prevent the aerosol generating material from remaining inside the aerosol generating device 10 as the aerosol generating material fails to reach the atomizer, or from coming into contact with other components arranged in the housing (e.g., the housing 100 of FIG. 1).

Part of the aerosol generating material may be discharged through the ruptured portion without passing through the hollow hole 131. In this case, the aerosol generating material may move along the external surface of the rupture unit 130b.

Referring to FIG. 3C, the rupture unit 130c may include a hollow hole 131c extending in a lengthwise direction of the rupture unit 130c. In this case, the expression ‘gradient of the hollow hole 131c’ may indicate that the interior of the hollow hole 131c protrudes towards the central axis of the rupture unit 130c as it approaches the lower portion of the rupture unit 130c.

Because of the gradient of the hollow hole 131c, the diameter of the entrance of the hollow hole 131c, through which the aerosol generating material flows, may be greater than that of the outlet of the hollow hole 131c, through which the aerosol generating material is discharged. The aerosol generating material released from the ruptured capsule may smoothly flow into the hollow hole 131c through the entrance of the hollow hole 131c.

Although not illustrated in FIG. 3C, like the rupture unit 130a of FIG. 3A, the external surface of the rupture unit 130c may be inclined in the lengthwise direction of the rupture unit 130c.

Referring to FIG. 3D, the rupture unit 130d may include at least one hollow hole 131 through which the aerosol generating material moves. For example, the hollow hole 131 may include a first hollow hole 1311 and a second hollow hole 1312, wherein the first hollow hole 1311 extends in a direction (e.g., the lengthwise direction of the rupture unit), and the second hollow hole 1312 extends in a direction (e.g., a direction crossing the lengthwise direction of the rupture unit) that is different from the direction.

After the rupture of the capsule, the aerosol generating material may flow into the first hollow hole 1311 of the rupture unit 130d. The aerosol generating material may move along the first hollow hole 1311 and the second hollow hole 1312 and may be discharged outside the rupture unit 130d.

Referring to FIG. 3D, the second hollow hole 1312 is connected to an end portion of the first hollow hole 1311. However, the arrangements of the first hollow hole and the second hollow hole are not limited thereto. The first hollow hole extends to the lower portion of the rupture unit 130d like the rupture unit of FIG. 3B such that the aerosol generating material may be released through the first hollow hole.

Also, FIG. 3D illustrates that one first hollow hole 1311 formed in the z-axis direction and two second hollow holes 1312 formed in the y-axis direction, but the number, size, and direction of the first hollow hole and the second hollow hole are not limited thereto.

The aerosol generating material may be discharged from the rupture unit 130d along the first hollow hole 1311 and the second hollow hole 1312 in different directions. For example, when a portion of the rupture unit 130d is inserted into the atomizer, the aerosol generating material may disperse inside the atomizer in different directions, according to the direction in which the aerosol generating material is discharged through the hollow hole 131. Accordingly, the aerosol generating material may be effectively supplied to the atomizer.

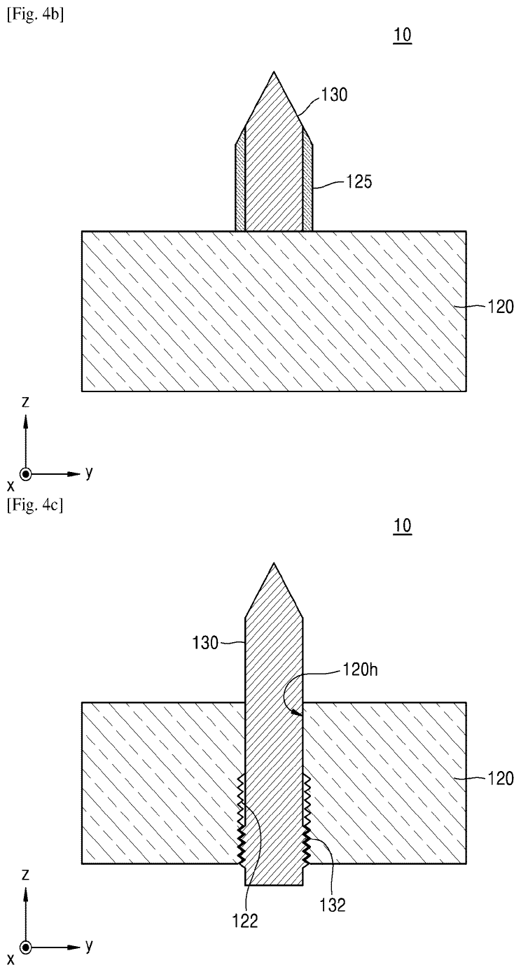

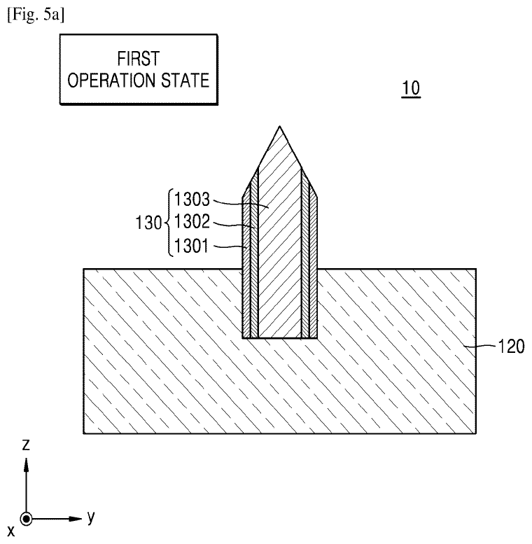

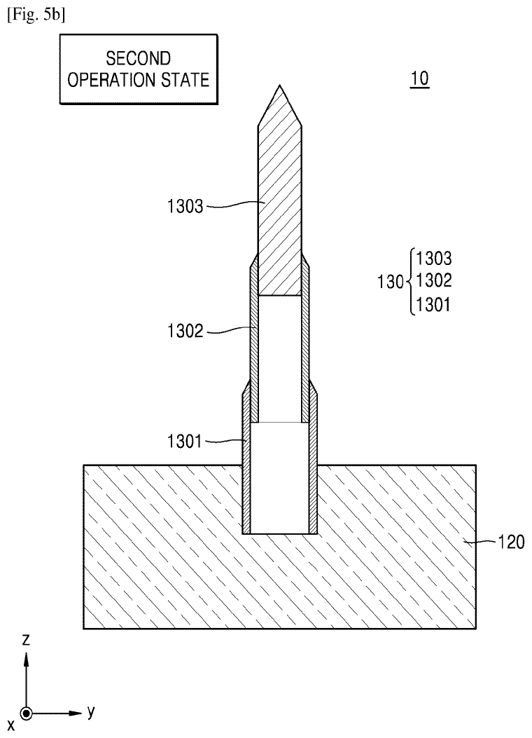

Hereinafter, with reference to FIGS. 4A to 4C, a method in which the rupture unit is coupled to the atomizer and the movement of the rupture unit are described.

FIGS. 4A to 4C respectively are cross-sectional views illustrating examples in which an atomizer and a rupture unit are coupled to each other in a structure applicable to the aerosol generating device according to the embodiment of FIG. 1.

Referring to FIGS. 4A to 4C, the aerosol generating device 10 according to an embodiment may include the atomizer 120 and the rupture unit 130.

The atomizer 120 and the rupture unit 130 respectively illustrated in FIGS. 4A to 4C may be the same as or similar to the atomizer 120 and the rupture unit 130 of FIG. 1, and repeated descriptions are omitted.

Referring to FIGS. 4A to 4C, the rupture unit 130 may be coupled to the atomizer 120. The method in which the rupture unit 130 is coupled to the atomizer 120 varies and is not limited to examples described below with reference to respective drawings.

At least a portion of the rupture unit 130 may move between the location in contact with the capsule (e.g., the capsule c of FIG. 1) and the location spaced apart from the capsule.

For example, the rupture unit 130 illustrated in FIGS. 4A and 4B may move together with the atomizer 120. In this case, the rupture unit 130 may be moved together with the atomizer 120 by a driver (not illustrated) connected to the atomizer 120.

As another example, although the rupture unit 130 of FIG. 4C may be coupled to the atomizer 120, the atomizer 120 may not move, and at least a portion of the rupture unit 130 may only move. However, the movement of the rupture unit 130 is not limited to the above examples.

Referring to FIG. 4A, the atomizer 120 may include an insertion groove 120g into which at least a portion of the rupture unit 130 is inserted. The insertion groove 120g may be open towards the location at which the capsule is ruptured. After the rupture unit 130 is inserted into the insertion groove 120g, the location at which the rupture occurs may be aligned with an extension of the lengthwise direction of the rupture unit 130.

The rupture unit 130 may be inserted into the insertion groove 120g in an interference fit manner and supported by the insertion groove 120g. However, the manner in which the rupture unit 130 is inserted into the insertion groove 120g is not limited thereto.

Referring to FIG. 4B, the aerosol generating device 10 may further include a delivery wick 125 arranged on at least a portion of the rupture unit 130 and configured to absorb the aerosol generating material. The delivery wick 125 may be a portion of the atomizer 120 or the rupture unit 130.

The delivery wick 125 may surround at least a portion of the rupture unit 130. The delivery wick 125 may contact the atomizer 120 and be connected thereto. For example, the delivery wick 125 may be connected to the liquid delivery element of the atomizer 120 and may realize a structure of a double wick. The delivery wick 125 may absorb the aerosol generating material released from the capsule ruptured by the rupture unit 130 and deliver the aerosol generating material to the atomizer 120.

When the delivery wick 125 is arranged on the circumference of the pointed end portion of the rupture unit 130, the delivery wick 125 may be inserted into the capsule together with the end portion of the rupture unit 130 and may absorb the aerosol generating material existing in the capsule.

The delivery wick 125 may contact the atomizer 120 and the rupture unit 130 and connect the same to each other. Accordingly, the rupture unit 130 may be coupled to the atomizer 120. For example, the rupture unit 130 may be inserted into the delivery wick 125 connected to the atomizer 120 and coupled to the atomizer 120. However, a manner in which the rupture unit 130 is coupled to the atomizer 120 by the delivery wick 125 is not limited to the above example.

Referring to FIG. 4C, the atomizer 120 may include a through hole 120h extending in a direction (e.g., the z-axis direction) such that at least a portion of the rupture unit 130 penetrates the atomizer 120.

The through hole 120h may be open towards the location at which the capsule is ruptured. When the rupture unit 130 is inserted into the through hole 120h, the location at which the capsule is ruptured may be aligned with the extension of the lengthwise direction of the rupture unit 130.

A female thread 122 may be formed in at least a portion of the through hole 120h. A male thread 132 corresponding to the female thread 122 of the through hole 120h may be formed in the outer side surface of the rupture unit 130. In this case, the locations of the female thread 122 and the male thread 132 may be interchangeable according to embodiments. That is, a male thread may be formed in the through hole 120h, and a female thread may be formed in the rupture unit 130.

The rupture unit 130 may be secured using the through hole 120h to move only in a direction in which the through hole 120h extends. Therefore, when the rupture unit 130 is rotated by the driver, the rupture unit 130 may rectilinearly move along the female thread 122 in the direction in which the through hole 120h extends.

Hereinafter, with reference to FIGS. 5A and 5B, an example in which only a portion of the rupture unit 130 moves without the movement of the atomizer 120 is described.

FIG. 5A is a cross-sectional view of an atomizer and a rupture unit, illustrating an example of the rupture unit in a first operation state. FIG. 5B is a cross-sectional view of an atomizer and a rupture unit, illustrating the rupture unit of FIG. 5A in a second operation state.

Referring to FIGS. 5A and 5B, the aerosol generating device 10 according to an embodiment may include the atomizer 120 and the rupture unit 130.

The atomizer 120 and the rupture unit 130 respectively illustrated in FIGS. 5A and 5B may be the same as or similar to the atomizer 120 and the rupture unit 130 of FIG. 1, and repeated descriptions are omitted.

Referring to FIGS. 5A and 5B, at least a portion of the rupture unit 130 may be inserted into the atomizer 120 and coupled thereto. In this case, the rupture unit 130 may be inserted into the atomizer 120 through an insertion groove (e.g., the insertion groove 120g of FIG. 4A) or a through hole (e.g., the through hole 120h of FIG. 4C) of the atomizer 120.

The rupture unit 130 may include a first portion 1301, a second portion 1302, and a third portion 1303. That is, the rupture unit 130 may include a three-stage structure including three portions.

At least a portion of the first portion 1301 may be arranged inside the atomizer 120 and coupled thereto. The first portion 1301 may be supported by the atomizer 120.

At least a portion of the second portion 1302 may be arranged inside the first portion 1301 and move in the lengthwise direction (e.g., the z-axis direction) of the rupture unit 130.

At least a portion of the third portion 1303 may be arranged inside the second portion 1302 and move in the lengthwise direction of the rupture unit 130, thereby rupturing the capsule (e.g., the capsule c of FIG. 1).

The second portion 1302 and the third portion 1303 may be respectively connected to the driver (not illustrated) and move in a straight line.

FIG. 5A illustrates that the second portion 1302 is inserted into the first portion 1301 and the third portion 1303 is inserted into the second portion 1302. Such a state is referred to as a first operation state of the rupture unit 130.

FIG. 5B illustrates that a portion of the second portion 1302 protrudes from the first portion 1301 and a portion of the third portion 1303 protrudes from the second portion 1302. Such a state is referred to as a second operation state of the rupture unit 130.

To rupture the capsule, the rupture unit 130 may be switched from the first operation state to the second operation state. That is, the second portion 1302 and the third portion 1303 may respectively move in the +z direction and approach the capsule.

The rupture unit 130 may be switched from the second operation state to the first operation state to be away from the ruptured capsule. That is, the second portion 1302 and the third portion 1303 may respectively move in the −z direction to be away from the capsule.

The first operation state and the second operation state are not limited to those illustrated in FIGS. 5A and 5B. Also, the rupture unit 130 is not limited to the three-stage structure of FIGS. 5A and 5B. For example, the rupture unit 130 may have a two-stage structure including only a first portion and a second portion. In this case, the second portion may rupture the capsule.

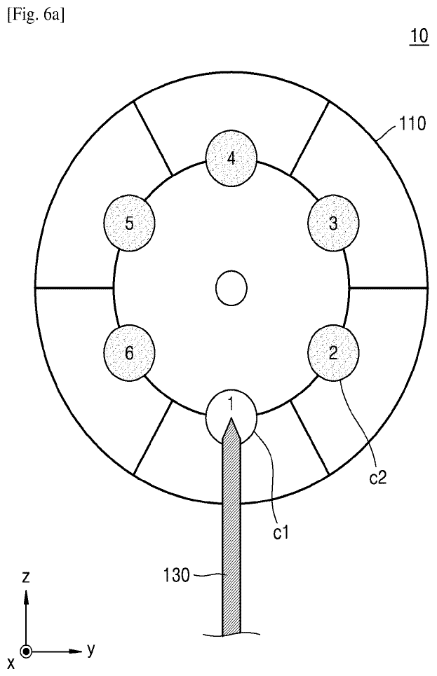

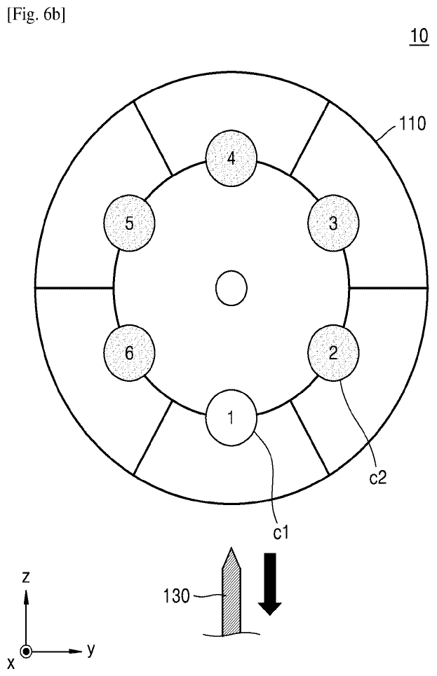

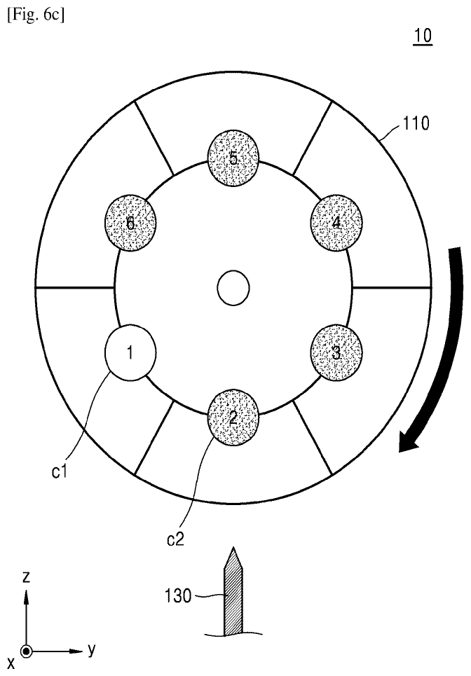

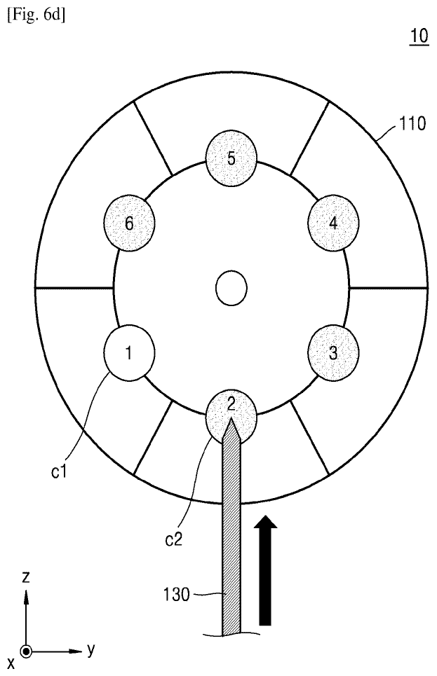

Hereinafter, with reference to FIGS. 6A to 6D, a capsule processing process by the storage and the rupture unit is described.

FIGS. 6A to 6D are cross-sectional views illustrating, in sequence, a capsule processing process of an aerosol generating device, according to an embodiment.

Referring to FIGS. 6A to 6D, the aerosol generating device 10 according to an embodiment may include the storage 110 and the rupture unit 130.

The storage 110 and the rupture unit 130 illustrated in FIGS. 6A to 6D may be the same as or similar to the storage 110 and the rupture unit 130 of FIG. 1, and repeated descriptions are omitted.

FIG. 6A illustrates that the rupture unit 130 ruptures a first capsule c1 mounted in the storage 110, and thus the first capsule c1 is empty.

Referring to FIG. 6A, a plurality of capsules may be mounted in the storage 110. The capsules mounted in the storage 110 may be arranged in sequence to form a circle.

Among the capsules, the first capsule c1 is arranged at the location at which the rupture occurs. The rupture unit 130 may rupture the first capsule c1 arranged at the location corresponding to the rupture unit 130. Accordingly, the first capsule c1 may be ruptured, while other capsules may remain stored.

The aerosol generating material released from the first capsule c1 may be supplied to the atomizer (e.g., the atomizer 120 of FIG. 1). The first capsule c1 may no longer store the aerosol generating material, and thus, the inside of the first capsule c1 may be empty.

After the rupture unit 130 ruptures the first capsule c1 and the aerosol generating material is completely released from the first capsule c1, the rupture unit 130 may remain inserted into the first capsule c1.

When the atomizer does not atomize the aerosol generating material into aerosols, the aerosol generating material may exist in the atomizer while supplied to the atomizer. In this case, the inside of the aerosol generating device 10 may be the same as the state illustrated in FIG. 6A.

When the user of the aerosol generating device 10 uses the aerosol generating device 10, that is, even when the atomizer atomizes the aerosol generating material into the aerosol, the inside of the aerosol generating device 10 may be the same as the state illustrated in FIG. 6A. The state of FIG. 6A may be maintained until most of the aerosol generating material supplied to the atomizer is atomized into the aerosol.

When most of the aerosol generating material supplied to the atomizer is atomized into the aerosol, there is a need to supply a new aerosol generating material to the atomizer. In this case, other capsules, mounted in the storage 110 but having not yet been ruptured, need to be ruptured. Accordingly, a series of the capsule processing process may be performed.

FIG. 6B illustrates that the rupture unit 130 is away from the ruptured first capsule c1.

Referring to FIG. 6B, to rupture other capsules, the rupture unit 130 may move in a direction opposite to the direction facing the ruptured first capsule c1 and be away from the ruptured first capsule c1. That is, the rupture unit 130 may move towards the location spaced apart from the first capsule c1.

FIG. 6C illustrates that the storage 110 rotates and then is aligned at a location, at which a second capsule c2 stored is ruptured.

Referring to FIG. 6C, the storage 110 may rotate around the central portion (e.g., the central portion 113 of FIG. 2A) of the storage 110 which is taken as the rotation axis. As the storage 110 rotates, the capsules may rotate and move with respect to the central portion.

The first capsule c1 at the location where the rupture occurs may be away from the rupture unit 130 along the trajectory of the capsule. The second capsule c2, which is adjacent to the first capsule c1, may come close to the location, where the rupture occurs, along the trajectory of the capsule. After the storage 110 finishes rotating, the second capsule c2 may be aligned at the location at which the rupture occurs.

Referring to FIG. 6C, the storage 110 may rotate 60 degrees in a clockwise direction, but the rotation direction and the angle of the storage 110 are not limited thereto. The rotation angle of the storage 110 may differ according to the number of capsules that may be maximally mounted in the storage 110.

FIG. 6D illustrates that the rupture unit 130 approaches the second capsule c2 and is inserted thereinto.

Referring to FIG. 6D, at least a portion of the rupture unit 130 may approach the second capsule c2 aligned at the location where the rupture occurs. That is, at least a portion of the rupture unit 130 may be moved towards the location at which the rupture unit 130 is in contact with the capsule.

The rupture unit 130 may rupture the second capsule c2 aligned at the location corresponding to the rupture unit 130. That is, the first capsule c1 and the second capsule c2 may be ruptured, while other capsules may remain stored.

Referring to FIG. 6D, the second capsule c2 may be ruptured by the rupture unit 130, but no aerosol generating material is released therefrom. However, one of ordinary skill in the art would easily understand that the aerosol generating material may be released from the ruptured second capsule c2.

A series of “capsule processing processes” performed in the state of FIG. 6A and described with reference to FIGS. 6B to 6D may include a series of sequential processes: a process in which the rupture unit 130 moves to a location spaced apart from the ruptured first capsule c1, a process in which the storage 110 rotates and moves to the location at which the second capsule c2 stored is ruptured, and a process in which the rupture unit 130 moves to the location in contact with the stored second capsule c2 and ruptures the second capsule c2.

When the aerosol generating material is released from the ruptured second capsule c2 and supplied to the atomizer, the user of the aerosol generating device 10 may use the aerosol generating device 10 again. That is, the capsule processing process may be performed after the user finishes using the aerosol generating device 10 and before the user uses the aerosol generating device 10 again.

In addition, through the capsule processing process, the capsules may be sequentially ruptured in an order in which the capsules are arranged in a circular form. The capsule processing process may be performed until all of the capsules in the storage 110 are ruptured.

When there is no capsule left because all of the capsules mounted are ruptured, the user needs to mount a new capsule in the storage 110. In this case, the capsule processing process may be performed to the state of FIG. 6B, but one or more embodiments are not limited thereto.

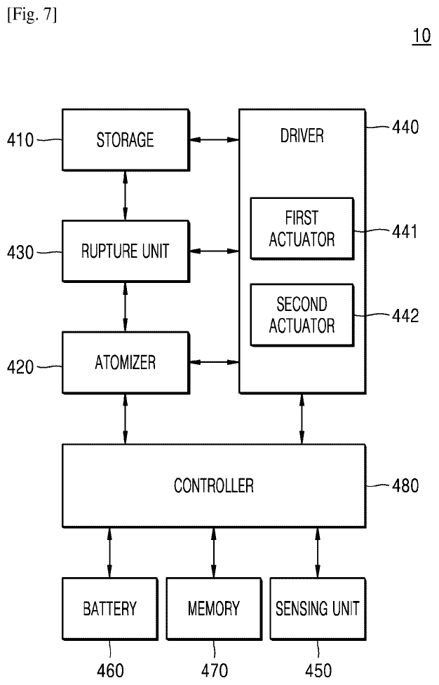

Hereinafter, with reference to FIG. 7, the control over the movement of the storage 110 and the rupture unit 130 is described.

FIG. 7 is a block diagram of an aerosol generating device according to an embodiment.

Referring to FIG. 7, the aerosol generating device 10 according to an embodiment may include a storage 410, an atomizer 420, a rupture unit 430, a driver 440, a sensing unit 450, a battery 460, a memory 470, and a controller 480.

The storage 410, the atomizer 420, and the rupture unit 430 of FIG. 7 may be the same as those of FIG. 1, and repeated descriptions are omitted.

The driver 440 may be connected to at least one of the storage 410, the atomizer 420, and the rupture unit 430 and move the storage 410 and/or the rupture unit 430.

The driver 440 may include one or more actuators. In this case, the actuator may include various configurations that perform mechanical works using electricity, hydraulic pressure, compressed air, or the like. For example, the actuator may include a motor. The actuator may perform a rectilinear motion as well as a rotary motion and thus may move components connected to the actuator rotationally and/or rectilinearly.

The driver 440 may include a first actuator 441 moving at least a portion of the storage 410, and a second actuator 442 moving at least a portion of the rupture unit 430.

The first actuator 441 may be connected to the storage 410. The first actuator 441 may rotate the storage 410 to align the capsule (e.g., the capsule c of FIG. 1), mounted in the storage 410, at the location corresponding to the rupture unit 430.

The second actuator 442 may be connected to the atomizer 420 or the rupture unit 430. When the second actuator 442 is connected to the atomizer 420, the second actuator 442 may move the atomizer 420 so that the atomizer 420 may move together with the rupture unit 430. When the second actuator 442 is connected to the rupture unit 430, the second actuator 442 may only move the rupture unit 430.

The second actuator 442 may move at least a portion of the rupture unit 430 in a straight line, between the location where the rupture unit 430 contacts the capsule, and the location where the rupture unit 430 is spaced apart from the capsule. For example, the second actuator 442 may move the rupture unit 430 in the lengthwise direction of the rupture unit 430.

The driver 440 may include one or more gears (not illustrated). The gears may be arranged separately from the actuators. The gear may connect the actuator to at least one of the storage 410, the atomizer 420, and the rupture unit 430.

The gear may not only reduce the speed of the actuator, but change the movement direction of the actuator according to the type of the actuator and change a rotary motion to a rectilinear motion.

The aerosol generating device 10 may use the sensing unit 450 to detect the existence of aerosol generating material in the atomizer 420. For example, because the sensing unit 450 may generate a signal based on a temperature of an atomizing element of the atomizer 420, when the aerosol generating material is exhausted and the atomizer 420 is heated to a high temperature, the existence of aerosol generating material may be detected based on the signal from the sensing unit 450.

As another example, the existence of aerosol generating material may be detected using the sensing unit 450 including a fixed resistor arranged in parallel with the atomizing element. In this case, the resistance value of the fixed resistor does not change according to the temperature of the atomizing element, and the fixed resistor may be arranged to only detect the existence of aerosol generating material.

Depending on whether the aerosol generating material absorbed into the liquid delivery element of the atomizer 420 exists, the temperature of the atomizing element in the liquid delivery element may change. In this case, when the atomizing element includes a resistor with a TCR, the intensity of resistance of the resistor may change according to the change in the temperature of the atomizing element. Therefore, a voltage difference in both ends of the resistor may differ.

Using the sensing unit 450 that generates a signal based on a voltage difference between both ends of the resistor or the fixed resistor of the atomizing element, the controller 480 may detect the existence of aerosol generating material in the atomizer 420.

In detail, by referencing a lookup table stored in the memory 470, the controller 480 may analyze result values corresponding to the voltage difference in both ends of the resistor and may determine whether the aerosol generating material exists.

When the existence of the aerosol generating material in the atomizer 420 is determined by the controller 480, the controller 480 may transmit, to the sensing unit 450, a signal including the result value regarding the existence of the aerosol generating material. In addition, the controller 480 may control other components of the aerosol generating device 10 based on a result regarding the existence of aerosol generating material.

The method of detecting the existence of the aerosol generating material is not limited to the above examples and may include various methods of detecting the existence of the aerosol generating material in the atomizer 420.

The sensing unit 450 may generate a signal according a change in the amount of aerosol generating material existing in the atomizer 420. For example, the sensing unit 450 may generate a signal with a size linearly changing according to the change in the amount of aerosol generating material existing in the atomizer 420.

As another example, the sensing unit 450 may generate a signal when the aerosol generating material existing in the atomizer 420 is reduced to a specific value or less. In this case, “specific value” refers to a reference value used to determine that the aerosol generating material does not exist in the atomizer 420 and may be a value that is set in advance in the memory 470.

The signal generated by the sensing unit 450 may be transmitted to the controller 480. Accordingly, the controller 480 may control the aerosol generating device 10 to enable other functions to work.

The battery 460 supplies power to be used for the aerosol generating device 10 to operate. That is, the battery 460 may supply power such that the atomizer 420 may be heated. In addition, the battery 460 may supply power required for operations of other components of the aerosol generating device 10, that is, the driver 440, the sensing unit 450, the memory 470, and the controller 480.

The memory 470 is a hardware component that stores various types of data processed by the aerosol generating device 10, and may store data processed and data to be processed by the controller 480. For example, in the memory 470, data (the above-described “look-up table”) associated with the existence of aerosol generating material in the atomizer may be stored.

The controller 480 may generally control operations of the aerosol generating device 10. In detail, the controller 480 controls operations of the components included in the aerosol generating device 10. Also, the controller 480 may check a state of each of the components of the aerosol generating device 10 to determine whether or not the aerosol generating device 10 is able to operate.

The controller 480 may sense the signal generated by the sensing unit 450. The controller 480 that senses the signal may control subsequent processes to be performed to provide the aerosol generating material to the atomizer 420.

For example, the controller 480 may control movement of at least one of the storage 410 and the rupture unit 430, based on the signal generated by the sensing unit 450. In detail, the controller 480 may be connected to the driver 440 and transmit, to the driver 440, a command for moving the storage 410 and/or the rupture unit 430.

As another example, the controller 480 may generate a notification signal and output the same to the outside of the aerosol generating device 10. Because of the notification signal, the user may confirm that the aerosol generating material in the atomizer 420 is exhausted. The user may enable the controller 480 to transmit, to the driver 440, the command for moving the storage 410 and/or the rupture unit 430 by performing operations, such as pressing a button (not illustrated).

To sum up, the controller 480 may sense the signal generated by the sensing unit 450 and then control the aerosol generating material to be supplied to the atomizer 420 automatically or according to the user's manipulation.

According to the command from the controller 480, the capsule processing process may proceed. Accordingly, another capsule storing therein the aerosol generating material may be ruptured, and the aerosol generating material may be supplied to the atomizer 420.

The controller 480 may control at least one of the storage 410 and the rupture unit 430 to remain in a stationary state for a certain period of time after the capsule is ruptured by the rupture unit 430. In this case, the expression “certain period of time” may refer to a period of time sufficient enough to allow the aerosol generating material to be entirely released from the capsule immediately after the rupture of the capsule and be supplied to the atomizer 420. According to the control of the controller 480, the storage 410 may not rotate for a certain period of time, and the rupture unit 430 may remain inserted into the capsule and may not rectilinearly move.

According to an embodiment, when the aerosol generating material may not freely released from the capsule after the capsule is ruptured, the capsule processing process may be prevented from proceeding in a state in which the aerosol generating material is not sufficiently supplied to the atomizer 420.

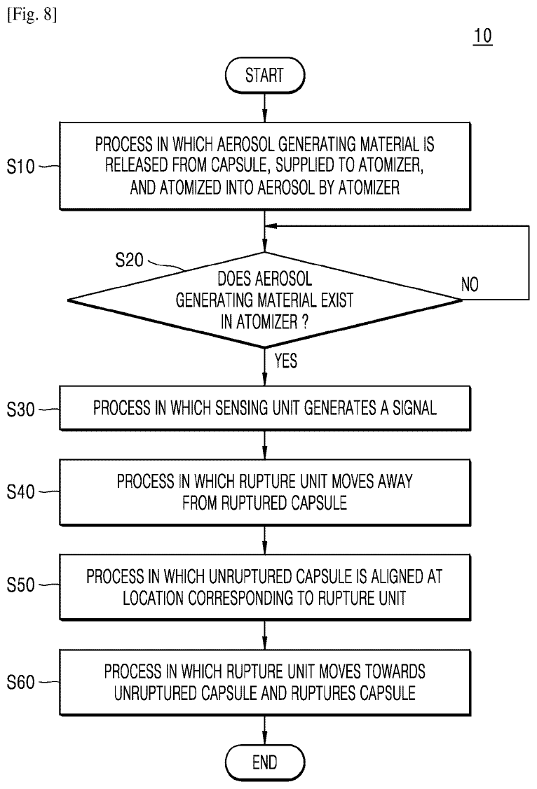

Hereinafter, with reference to FIG. 8, the capsule processing process is described based on the sensing unit and the controller.

FIG. 8 is a flowchart illustrating a capsule processing process of an aerosol generating device, according to an embodiment.

Hereinafter, for the explanation of the capsule processing process of FIG. 8, the components of the aerosol generating device 10 of FIGS. 1 and 7 are being referred to.

Referring to FIG. 8, according to the capsule processing process of the aerosol generating device 10 according to an embodiment, one or more unruptured capsules are mounted in the storage 110. Also, the capsule processing process of FIG. 8 starts from the state in which the capsule is ruptured by the rupture unit 130.

In operation S10, the aerosol generating material may be released from the capsule and supplied to the atomizer 120, thus being atomized into an aerosol. In this case, the user may inhale the aerosol by using the aerosol generating device 10.

In operation S20, the controller 480 may detect the existence of aerosol generating material in the atomizer 120, based on the signal of the sensing unit 450. When the controller 480 determines, based on the signal of the sensing unit 450, that the aerosol generating material exists in the atomizer 120, operation S20 may be performed again. When the controller 480 determines, based on the signal of the sensing unit 450, that the aerosol generating material does not exist in the atomizer 120, operation S30 may be performed.

In operation S30, the sensing unit 450 may generate a signal such that the controller 480 may control the movement of at least one of the storage 410 and the rupture unit 430.

Operation S30 may proceed when, for example, the controller 480 transmits to the sensing unit 450 a signal including a result value regarding the existence of aerosol generating material. In operation S20, when the controller 480 determines whether the aerosol generating material exists and then may immediately control other components (e.g., the storage 110 and the rupture unit 130) of the aerosol generating device 10 based on the result of determining, operation S30 may be omitted, and operation S40 may be performed.

In operation S40, the rupture unit 130 may move (e.g., rectilinearly move) in a direction away from the ruptured capsule. In this case, the controller 480 may control the movement of the rupture unit 130.

In operation S50, the storage 110 may move (e.g., rotate and move) an unruptured capsule to be aligned at the location corresponding to the rupture unit 130. In this case, the controller 480 may control the movement of the storage 110.

In operation S60, the rupture unit 130 may move (e.g., rectilinearly move) towards the unruptured capsule to rupture the capsule. In this case, the controller 480 may control the rectilinear motion of the rupture unit 130.

Operations S10 to S60 may be repeatedly performed until all capsules mounted in the storage 110 are ruptured and replaced with new capsules.

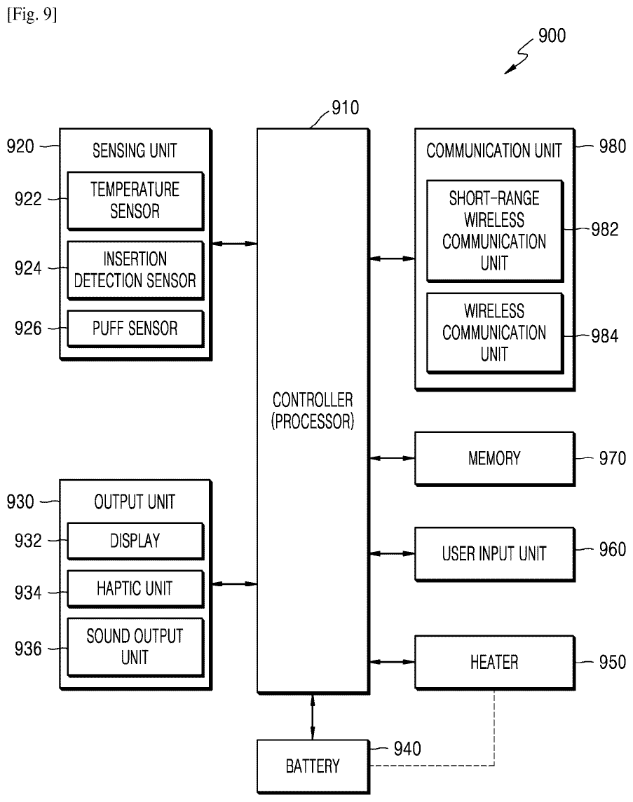

FIG. 9 is a block diagram of an aerosol generating device 900 according to another embodiment.

The aerosol generating device 900 may include a controller 910, a sensing unit 920, an output unit 930, a battery 940, a heater 950, a user input unit 960, a memory 970, and a communication unit 980. However, the internal structure of the aerosol generating device 900 is not limited to those illustrated in FIG. 9. That is, according to the design of the aerosol generating device 900, it will be understood by one of ordinary skill in the art that some of the components shown in FIG. 9 may be omitted or new components may be added.

The sensing unit 920 may sense a state of the aerosol generating device 900 and a state around the aerosol generating device 900, and transmit sensed information to the controller 910. Based on the sensed information, the controller 910 may control the aerosol generating device 900 to perform various functions, such as controlling an operation of the heater 950, limiting smoking, determining whether an aerosol generating article (e.g., a cigarette, a cartridge, or the like) is inserted, displaying a notification, or the like.

The sensing unit 920 may include at least one of a temperature sensor 922, an insertion detection sensor 924, and a puff sensor 926, but is not limited thereto.

The temperature sensor 922 may sense a temperature at which the heater 950 (or an aerosol generating material) is heated. The aerosol generating device 900 may include a separate temperature sensor for sensing the temperature of the heater 950, or the heater 950 may serve as a temperature sensor. Alternatively, the temperature sensor 922 may also be arranged around the battery 940 to monitor the temperature of the battery 940.

The insertion detection sensor 924 may sense insertion and/or removal of an aerosol generating article. For example, the insertion detection sensor 924 may include at least one of a film sensor, a pressure sensor, an optical sensor, a resistive sensor, a capacitive sensor, an inductive sensor, and an infrared sensor, and may sense a signal change according to the insertion and/or removal of an aerosol generating article.

The puff sensor 926 may sense a user's puff on the basis of various physical changes in an airflow passage or an airflow channel. For example, the puff sensor 926 may sense a user's puff on the basis of any one of a temperature change, a flow change, a voltage change, and a pressure change.

The sensing unit 920 may include, in addition to the temperature sensor 922, the insertion detection sensor 924, and the puff sensor 926 described above, at least one of a temperature/humidity sensor, a barometric pressure sensor, a magnetic sensor, an acceleration sensor, a gyroscope sensor, a location sensor (e.g., a global positioning system (GPS)), a proximity sensor, and a red-green-blue (RGB) sensor (illuminance sensor). Because a function of each of sensors may be intuitively inferred by one of ordinary skill in the art from the name of the sensor, a detailed description thereof may be omitted.

The output unit 930 may output information on a state of the aerosol generating device 900 and provide the information to a user. The output unit 930 may include at least one of a display unit 932, a haptic unit 934, and a sound output unit 936, but is not limited thereto. When the display unit 932 and a touch pad form a layered structure to form a touch screen, the display unit 932 may also be used as an input device in addition to an output device.

The display unit 932 may visually provide information about the aerosol generating device 900 to the user. For example, information about the aerosol generating device 900 may mean various pieces of information, such as a charging/discharging state of the battery 940 of the aerosol generating device 900, a preheating state of the heater 950, an insertion/removal state of an aerosol generating article, or a state in which the use of the aerosol generating device 900 is restricted (e.g., sensing of an abnormal object), or the like, and the display unit 932 may output the information to the outside. The display unit 932 may be, for example, a liquid crystal display panel (LCD), an organic light-emitting diode (OLED) display panel, or the like. In addition, the display unit 932 may be in the form of a light-emitting diode (LED) light-emitting device.

The haptic unit 934 may tactilely provide information about the aerosol generating device 900 to the user by converting an electrical signal into a mechanical stimulus or an electrical stimulus. For example, the haptic unit 934 may include a motor, a piezo-electric element, or an electrical stimulation device.

The sound output unit 936 may audibly provide information about the aerosol generating device 900 to the user. For example, the sound output unit 936 may convert an electrical signal into a sound signal and output the same to the outside.

The battery 940 may supply power used to operate the aerosol generating device 900. The battery 940 may supply power such that the heater 950 may be heated. In addition, the battery 940 may supply power required for operations of other components (e.g., the sensing unit 920, the output unit 930, the user input unit 960, the memory 970, and the communication unit 980) in the aerosol generating device 900. The battery 940 may be a rechargeable battery or a disposable battery. For example, the battery 940 may be a lithium polymer (LiPoly) battery, but is not limited thereto.

The heater 950 may receive power from the battery 940 to heat an aerosol generating material. Although not illustrated in FIG. 9, the aerosol generating device 900 may further include a power conversion circuit (e.g., a direct current (DC)/DC converter) that converts power of the battery 940 and supplies the same to the heater 950. In addition, when the aerosol generating device 900 generates aerosols in an induction heating method, the aerosol generating device 900 may further include a DC/alternating current (AC) that converts DC power of the battery 940 into AC power.

The controller 910, the sensing unit 920, the output unit 930, the user input unit 960, the memory 970, and the communication unit 980 may each receive power from the battery 940 to perform a function. Although not illustrated in FIG. 9, the aerosol generating device 900 may further include a power conversion circuit that converts power of the battery 940 to supply the power to respective components, for example, a low dropout (LDO) circuit, or a voltage regulator circuit.

In an embodiment, the heater 950 may be formed of any suitable electrically resistive material. For example, the suitable electrically resistive material may be a metal or a metal alloy including titanium, zirconium, tantalum, platinum, nickel, cobalt, chromium, hafnium, niobium, molybdenum, tungsten, tin, gallium, manganese, iron, copper, stainless steel, nichrome, or the like, but is not limited thereto. In addition, the heater 950 may be implemented by a metal wire, a metal plate on which an electrically conductive track is arranged, a ceramic heating element, or the like, but is not limited thereto.

In another embodiment, the heater 950 may be a heater of an induction heating type. For example, the heater 950 may include a susceptor that heats an aerosol generating material by generating heat through a magnetic field applied by a coil.

The user input unit 960 may receive information input from the user or may output information to the user. For example, the user input unit 960 may include a key pad, a dome switch, a touch pad (a contact capacitive method, a pressure resistance film method, an infrared sensing method, a surface ultrasonic conduction method, an integral tension measurement method, a piezo effect method, or the like), a jog wheel, a jog switch, or the like, but is not limited thereto. In addition, although not illustrated in FIG. 9, the aerosol generating device 900 may further include a connection interface, such as a universal serial bus (USB) interface, and may connect to other external devices through the connection interface, such as the USB interface, to transmit and receive information, or to charge the battery 940.

The memory 970 is a hardware component that stores various types of data processed in the aerosol generating device 900, and may store data processed and data to be processed by the controller 910. The memory 970 may include at least one type of storage medium from among a flash memory type, a hard disk type, a multimedia card micro type memory, a card-type memory (for example, secure digital (SD) or extreme digital (XD) memory, etc.), random access memory (RAM), static random access memory (SRAM), read-only memory (ROM), electrically erasable programmable read-only memory (EEPROM), programmable read-only memory (PROM), a magnetic memory, a magnetic disk, and an optical disk. The memory 970 may store an operation time of the aerosol generating device 900, the maximum number of puffs, the current number of puffs, at least one temperature profile, data on a user's smoking pattern, etc.

The communication unit 980 may include at least one component for communication with another electronic device. For example, the communication unit 980 may include a short-range wireless communication unit 982 and a wireless communication unit 984.

The short-range wireless communication unit 982 may include a Bluetooth communication unit, a Bluetooth Low Energy (BLE) communication unit, a near field communication unit, a wireless LAN (WLAN) (Wi-Fi) communication unit, a Zigbee communication unit, an infrared data association (IrDA) communication unit, a Wi-Fi Direct (WFD) communication unit, an ultra-wideband (UWB) communication unit, an Ant+ communication unit, or the like, but is not limited thereto.

The wireless communication unit 984 may include a cellular network communication unit, an Internet communication unit, a computer network (e.g., local area network (LAN) or wide area network (WAN)) communication unit, or the like, but is not limited thereto. The wireless communication unit 984 may also identify and authenticate the aerosol generating device 900 within a communication network by using subscriber information (e.g., International Mobile Subscriber Identifier (IMSI)).

The controller 910 may control general operations of the aerosol generating device 900. In an embodiment, the controller 910 may include at least one processor. The processor may be implemented as an array of a plurality of logic gates or may be implemented as a combination of a general-purpose microprocessor and a memory in which a program executable by the microprocessor is stored. It will be understood by one of ordinary skill in the art that the processor may be implemented in other forms of hardware.

The controller 910 may control the temperature of the heater 950 by controlling supply of power of the battery 940 to the heater 950. For example, the controller 910 may control power supply by controlling switching of a switching element between the battery 940 and the heater 950. In another example, a direct heating circuit may also control power supply to the heater 950 according to a control command of the controller 910.

The controller 910 may analyze a result sensed by the sensing unit 920 and control subsequent processes to be performed. For example, the controller 910 may control power supplied to the heater 950 to start or end an operation of the heater 950 on the basis of a result sensed by the sensing unit 920. As another example, the controller 910 may control, based on a result sensed by the sensing unit 920, an amount of power supplied to the heater 950 and the time the power is supplied, such that the heater 950 may be heated to a certain temperature or maintained at an appropriate temperature.

The controller 910 may control the output unit 930 on the basis of a result sensed by the sensing unit 920. For example, when the number of puffs counted through the puff sensor 926 reaches a preset number, the controller 910 may notify the user that the aerosol generating device 900 will soon be terminated through at least one of the display unit 932, the haptic unit 934, and the sound output unit 936.

One embodiment may also be implemented in the form of a computer-readable recording medium including instructions executable by a computer, such as a program module executable by the computer. The computer-readable recording medium may be any available medium that may be accessed by a computer and includes both volatile and nonvolatile media, and removable and non-removable media. In addition, the computer-readable recording medium may include both a computer storage medium and a communication medium. The computer storage medium includes all of volatile and nonvolatile media, and removable and non-removable media implemented by any method or technology for storage of information such as computer-readable instructions, data structures, program modules, or other data. The communication medium typically includes computer-readable instructions, data structures, other data in modulated data signals such as program modules, or other transmission mechanisms, and includes any information transfer media.

The descriptions of the above-described embodiments are merely examples, and it will be understood by one of ordinary skill in the art that various changes and equivalents thereof may be made. Therefore, the scope of the disclosure should be defined by the appended claims, and all differences within the scope equivalent to those described in the claims will be construed as being included in the scope of protection defined by the claims.

1. An aerosol generating device comprising:
a storage comprising a plurality of capsules storing an aerosol generating material;
an atomizer configured to generate an aerosol from the aerosol generating material; and
a rupture unit configured to rupture the capsules and supply the aerosol generating material stored in the capsules to the atomizer,
wherein the storage is movable such that the capsules are aligned at a location corresponding to the rupture unit.

2. The aerosol generating device of claim 1, wherein the storage is rotatable.

3. The aerosol generating device of claim 1, wherein at least a portion of the rupture unit is inclined in a direction in which the rupture unit extends.

4. The aerosol generating device of claim 1, wherein the rupture unit comprises a hollow hole through which the aerosol generating material moves.

5. The aerosol generating device of claim 4, wherein the hollow hole comprises a first hollow hole extending in a lengthwise direction of the rupture unit, and a second hollow hole connected to the first hollow hole and extending in a direction crossing the lengthwise direction of the rupture unit.

6. The aerosol generating device of claim 1, wherein at least a portion of the rupture unit is movable between a location in contact with the capsule and a location spaced apart from the capsule.

7. The aerosol generating device of claim 1, wherein the rupture unit is coupled to the atomizer and movable together with the atomizer.

8. The aerosol generating device of claim 1, wherein the atomizer comprises an insertion groove for supporting the rupture unit by accommodating at least a portion of the rupture unit.

9. The aerosol generating device of claim 1, further comprising a delivery wick arranged on at least a portion of the rupture unit and configured to absorb the aerosol generating material.

10. The aerosol generating device of claim 1, wherein the rupture unit comprises a first portion, which is coupled to and supported by the atomizer, and a second portion, at least a portion of which is arranged on the first portion and which is moved in a lengthwise direction of the rupture unit to rupture the capsules.

11. The aerosol generating device of claim 1, further comprising a driver configured to move the storage to align the capsules at the location corresponding to the rupture unit.

12. The aerosol generating device of claim 11, wherein the driver is configured to move the rupture unit in the lengthwise direction of the rupture unit.

13. The aerosol generating device of claim 1, further comprising a sensing unit configured to generate a signal according to a change in an amount of aerosol generating material present in the atomizer.

14. The aerosol generating device of claim 13, further comprising a controller configured to control operation of the storage, wherein the controller is configured to control movement of the storage, based on the signal generated by the sensing unit.

15. The aerosol generating device of claim 14, wherein the controller is configured to control at least one of the storage and the rupture unit to remain in a stationary state for a certain period of time after the rupture unit ruptures the capsules.
